(19) United States
(12) Patent Application Publication
(10) Pub. No.: US 2021/0072222 A1
(43) Pub. Date: Mar. 11, 2021

(54) COMPOSITIONS AND METHODS FOR VISUALIZATION OF EXTRACELLULAR ACTIVITY

(71) Applicant: University of Massachusetts, Boston, MA (US)

(72) Inventors: William R. Kobertz, Framingham, MA (US); Mei Zhang, Worcester, MA (US)

(21) Appl. No.: 17/014,397

(22) Filed: Sep. 8, 2020

Related U.S. Application Data
(60) Provisional application No. 62/896,524, filed on Sep. 5, 2019.

(51) Int. Cl. G01N 33/487; C07K 14/415; G01N 27/333; G01N 33/533

(52) U.S. Cl. CPC G01N 33/48721; C07K 14/415; G01N 33/533; G01N 27/333; G01N 33/4875

(57) ABSTRACT

The invention provides novel potassium ion sensor conjugates for spatiotemporal visualization of ion channel and membrane transporter activities from mammalian and human cells, and compositions and methods thereof.

### PRIORITY CLAIMS AND RELATED APPLICATIONS

This application claims the benefit of priority to U.S. Provisional Application No. 62/896,524, filed Sep. 5, 2019, the entire content of which is incorporated herein by reference for all purposes.

### STATEMENT REGARDING FEDERALLY FUNDED RESEARCH OR DEVELOPMENT

This invention was made with government support under grant number GM070650 awarded by the National Institutes of Health. The Government has certain rights in the invention.

### TECHNICAL FIELDS OF THE INVENTION

The invention generally relates to compounds and methods useful in detection and measurement of biological processes. More particularly, the invention provides novel potassium ion sensor conjugates for spatiotemporal visualization of ion channel and membrane transporter activities from mammalian and human cells, and compositions and methods thereof.

### BACKGROUND OF THE INVENTION

Potassium ion (K⁺) levels play significant roles in biological samples, for example, in various cellular and extracellular processes. Accurate imaging, assessment and measurement of K⁺ concentration in vivo or in vitro of K⁺ levels and activates is of great interest, in particular the spatial and temporal real-time determination of K⁺ activity and concentration.

The transverse (t)-tubules are a labyrinth of invaginations in the sarcolemma of ventricular myoctyes with intricate membrane folds that transverse and protrude in several directions. Myocytes from patients with cardiomyopathies and ischemic heart failure all have disrupted t-tubule networks. However, even modest t-tubule remodeling—loss of the membrane folds that restrict ion diffusion—leads to a prolonged action potential duration and an increased susceptibility to ventricular arrhythmias. It has long been hypothesized that K⁺ accumulates faster in the t-tubules to protect the heart during physical activity; however, current methodologies cannot measure ion concentrations in these extracellular microdomains.

There is an urgent need for development of improved methods to measure K⁺ activity and concentration in biological samples. In particular there is a need for methods to measure K⁺ concentration under physiological conditions in living systems.

### SUMMARY OF THE INVENTION

The invention provides novel fluorescent WGA-K⁺ sensor conjugates. The WGA-K⁺ sensor conjugates disclosed herein are capable of labeling both t-tubule and sarcolemma glycocalyces, enabling the spatiotemporal imaging of K⁺ accumulation over the entire cell surface of a living cardiomyocyte. Fluorescent WGA-K⁺ sensor conjugates of the invention enable spatiotemporal visualization of extracellular ion accumulation. The covalent attachment of fluorescent ion sensors to the outer vestibule of an ion channel allows detection of specific ion fluxes.

In one aspect, the invention generally relates to a compound comprising Wheat Germ Agglutinin (WGA) covalently conjugated to a potassium ion (K⁺) sensor:

WGA-(K⁺sensor)ₙ,

wherein n is an integer from 1 or 2.

In another aspect, the invention generally relates to a composition comprising a WGA-(K⁺ sensor)ₙ compound disclosed herein.

In yet another aspect, the invention generally relates to a kit for the assessing extracellular potassium ion, the kit comprising a WGA-(K⁺ sensor)ₙ compound disclosed herein.

In yet another aspect, the invention generally relates to a method for making a WGA-(K⁺ sensor)ₙ compound disclosed herein. The method comprises reacting WGA with a precursor of the K⁺ sensor to form a covalent linkage therebetween.

In yet another aspect, the invention generally relates to a complex comprising a WGA-(K⁺ sensor)ₙ compound complexed to a K⁺.

In yet another aspect, the invention generally relates to a composition comprising a K⁺ complex disclosed herein.

In yet another aspect, the invention generally relates to a method for imaging potassium ion (K⁺). The method comprises: contacting a sample of cells or tissue with a compound comprising Wheat Germ Agglutinin (WGA) covalently conjugated to a fluorescent potassium ion (K⁺) sensor; and acquiring one or more fluorescent images of the sample.

### DETAILED DESCRIPTION OF THE INVENTION

The invention is based in part on the discovery of novel fluorescent WGA-K⁺ sensor conjugates, which can be utilized for spatiotemporal imaging of K⁺ accumulation over the entire cell surface of a living cardiomyocyte. Because extracellular K⁺ accumulation arises from an increase in electrical activity, the fluorescence response is much slower (seconds) than membrane voltage changes or intracellular calcium transients, which makes imaging the physiological phenomenon of K⁺ accumulation tractable.

Fluorescent WGA-K⁺ sensor conjugates of the invention enable spatiotemporal visualization of extracellular ion accumulation. The covalent attachment of fluorescent ion sensors to the outer vestibule of an ion channel allows detection of specific ion fluxes.

Spatiotemporal visualization of extracellular K⁺ using the WGA-K⁺ sensor conjugates disclosed herein are suitable for studying various mammalian cells, including human tissue, since the disclosed approach is not genetically-encoded.

In one aspect, the invention generally relates to a compound comprising Wheat Germ Agglutinin (WGA) covalently conjugated to a potassium ion (K⁺) sensor:

WGA-(K⁺sensor)ₙ,

wherein n is an integer from 1 or 2.

In certain embodiments, n is 1. In certain embodiments, n is 2.

In certain embodiments, the K⁺ sensor comprises a fluorophore adapted to exhibiting a change in fluorescent profile and/or intensity upon binding of the sensor to a potassium ion. In certain embodiments, the fluorescent profile comprises an emission in the range from about 500 nm to about 600 nm (e.g., from about 520 nm to about 560 nm). In certain embodiments, the excitation comprises a wavelength at about 488 nm.

In certain embodiments, the K⁺ sensor comprises an ionophore capable of complexing a potassium ion.

WGA may be covalently linked to the potassium ion sensor via either the ionophore or the fluorophore. In certain embodiments, WGA is covalently conjugated to the fluorophore. In certain embodiments, WGA is covalently conjugated to the ionophore.

WGA may be covalently linked to the potassium ion sensor via any suitable bond or linkage, either directly or through a linker or spacer. In certain embodiments, WGA is covalently linked to the potassium ion sensor directly via a bond. In certain embodiments, WGA is covalently linked to the potassium ion sensor via a linker or spacer. In certain embodiments, WGA is covalently conjugated to the potassium ion sensor via an ester bond. In certain embodiments, WGA is covalently conjugated to the potassium ion sensor via an amide bond.

Any suitable fluorophores may be utilized, for example, xanthene derivatives, cyanine derivatives, coumarin derivatives, acridine derivatives, etc.

In certain embodiments, the fluorophore of the K⁺ sensor comprises the following conjugated system:

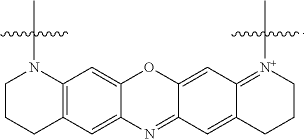 [SMILES: *N1CCCc2cc3c(cc21)Oc1cc2c(cc1=N3)CCC[N+]=2*]

In certain embodiments, the fluorophore of the K⁺ sensor comprises:

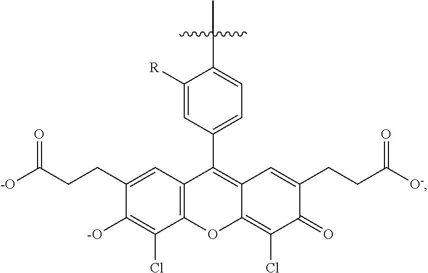

wherein R is a C₁-C₆ alkyl or a C₁-C₆ alkoxy group.

Any suitable ionophores may be employed. In certain embodiments, the ionophore is a crown ether or a derivative thereof. In certain embodiments, the ionophore is a cryptand or a derivative thereof (See, e.g., U.S. Pat. No. 6,211,359 B2; Lehn et al. 19751 Amer. Chem. Soc. 97:6700-6207.)

In certain embodiments, the ionophore comprises:

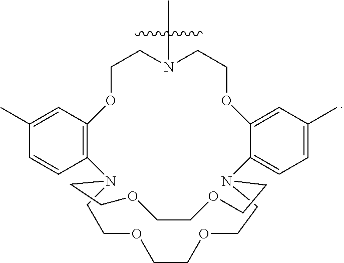

In certain embodiments, the ionophore comprises:

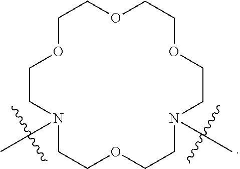 [SMILES: *N1CCOCCOCCOCCN(*)CCOCC1]

A non-limiting example of a K⁺ sensor having an ionophore and a fluorophore is

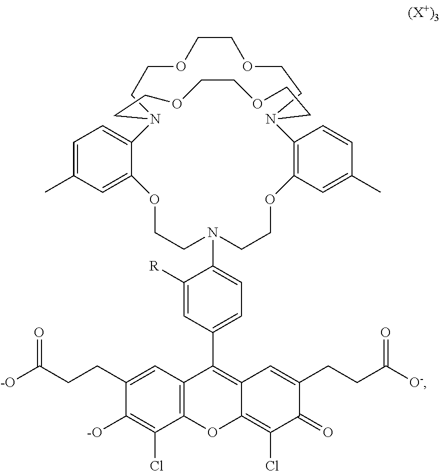

wherein X is a counterion, or a derivative thereof.

In another aspect, the invention generally relates to a composition comprising a WGA-(K⁺ sensor)ₙ compound disclosed herein.

In yet another aspect, the invention generally relates to a kit for the assessing extracellular potassium ion, the kit comprising a WGA-(K⁺ sensor)ₙ compound disclosed herein.

In yet another aspect, the invention generally relates to a method for making a WGA-(K⁺ sensor)ₙ compound disclosed herein. The method comprises reacting WGA with a precursor of the K⁺ sensor to form a covalent linkage therebetween.

In yet another aspect, the invention generally relates to a complex comprising a WGA-sensor)ₙ compound complexed to a K.

In yet another aspect, the invention generally relates to a composition comprising a K⁺ complex disclosed herein.

In yet another aspect, the invention generally relates to a method for imaging potassium ion (K⁺). The method comprises: contacting a sample of cells or tissue with a compound comprising Wheat Germ Agglutinin (WGA) covalently conjugated to a fluorescent potassium ion (K⁺) sensor; and acquiring one or more fluorescent images of the sample.

Any suitable biological cell samples or tissues may be imaged or analyzed according to the disclosed method. In certain embodiments, the sample is a sample mammalian cells or tissue. In certain embodiments, the sample is a sample human cells or tissue.

In certain embodiments of the method, the method comprises spatiotemporal visualization or imaging of activity of K⁺. In certain embodiments, the method comprises spatiotemporal visualization or imaging of activity of ion channel and membrane transportation.

In certain embodiments, the method further comprises assessing and/or measuring K⁺ concentration.

In certain embodiments of the method, the compound comprising WGA has the formula:

WGA-(K⁺sensor)ₙ,

wherein n is an integer from 1 or 2.

In certain embodiments of the method, the K⁺ sensor comprises a fluorophore adapted to exhibiting a change in fluorescent profile and/or intensity upon binding of the sensor to a potassium ion. In certain embodiments, the fluorescent profile comprises an emission in the range from about 500 nm to about 600 nm (e.g., from about 520 nm to about 560 nm).

In certain embodiments of the method, the K⁺ sensor comprises an ionophore capable of complexing a potassium ion.

WGA may be covalently linked to the potassium ion sensor via either the ionophore or the fluorophore. In certain embodiments, WGA is covalently conjugated to the fluorophore. In certain embodiments, WGA is covalently conjugated to the ionophore.

WGA may be covalently linked to the potassium ion sensor via any suitable bond or linkage, either directly or through a linker or spacer. In certain embodiments, WGA is covalently linked to the potassium ion sensor directly via a bond. In certain embodiments, WGA is covalently linked to the potassium ion sensor via a linker or spacer. In certain embodiments, WGA is covalently conjugated to the potassium ion sensor via an ester bond. In certain embodiments, WGA is covalently conjugated to the potassium ion sensor via an amide bond.

Any suitable fluorophores may be utilized, for example, xanthene derivatives, cyanine derivatives, coumarin derivatives, acridine derivatives, etc.

In certain embodiments of the method, the fluorophore of the K⁺ sensor comprises the following conjugated system:

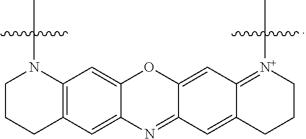 [SMILES: *N1CCCc2cc3c(cc21)Oc1cc2c(cc1=N3)CCC[N+]=2*]

In certain embodiments of the method, the fluorophore of the K⁺ sensor comprises:

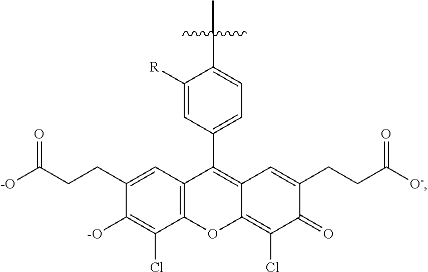

wherein R is a C₁-C₆ alkyl or a C₁-C₆ alkoxy group.

Any suitable ionophores may be employed. In certain embodiments, the ionophore is a crown ether or a derivative thereof. In certain embodiments, the ionophore is a cryptand or a derivative thereof.

In certain embodiments of the method, the ionophore comprises:

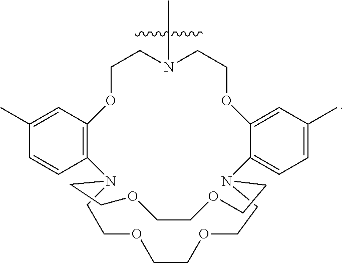

In certain embodiments of the method, the ionophore comprises:

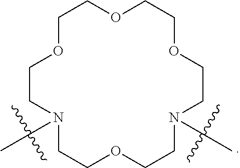 [SMILES: *N1CCOCCOCCOCCN(*)CCOCC1]

A non-limiting example of a K⁺ sensor having an ionophore and a fluorophore is

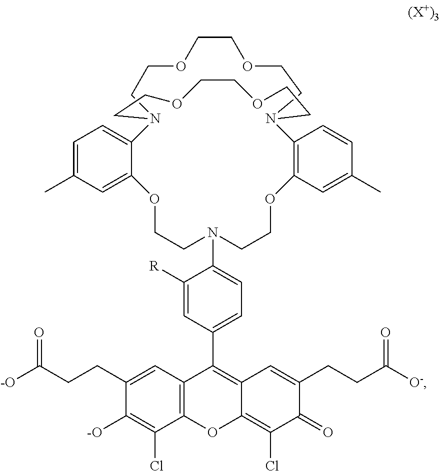

wherein X is a counterion, or a derivative thereof.

### Examples

Synthesis of WGA-K⁺ sensor-conjugate: IPG-4 (1.0 mg, 0.8 μmol) was reacted with 8 μmol of N,N,N′,N-Tetramethyl-O—(N-succinimidyl) uronium tetrafluoroborate (TSTU) in 200 μL of anhydrous N,N-dimethylformamide (DMF) at RT for 1 hr. The above reaction mixture was added to 1 mg of WGA (Vector Laboratories) in 1 mL of 0.1 M sodium bicarbonate (pH 8.3), containing 120 mg N-acetylglucosamine. The reaction mixture was stirred at RT for 2 hr and was terminated by adding 6.25 μL of a hydroxylamine solution to a final concentration of 0.1 M. The WGA-conjugate was separated from unreacted fluorophore by gravity gel filtration on a Sephadex G-25 Fine (20-50 μm) column equilibrated in PBS, final samples were lyophilized down to a powder. The degree of labeling (D.O.L.) was calculated using the following equation: the absorbance of WGA-conjugate was measured at 280 nm (A280) and the λₘₐₓ (λₘₐₓ).

$$DOL = \frac{{A}_{max} \times MW}{[ W G A ] \times {ɛ}_{dye}}$$

where MW is the molecular weight of WGA, ε_{dye} is the extinction coefficient of the dye at its absorbance maximum, and the WGA protein concentration is in mg/mL.

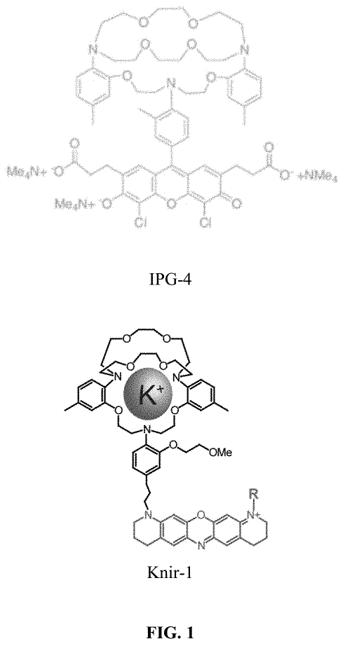

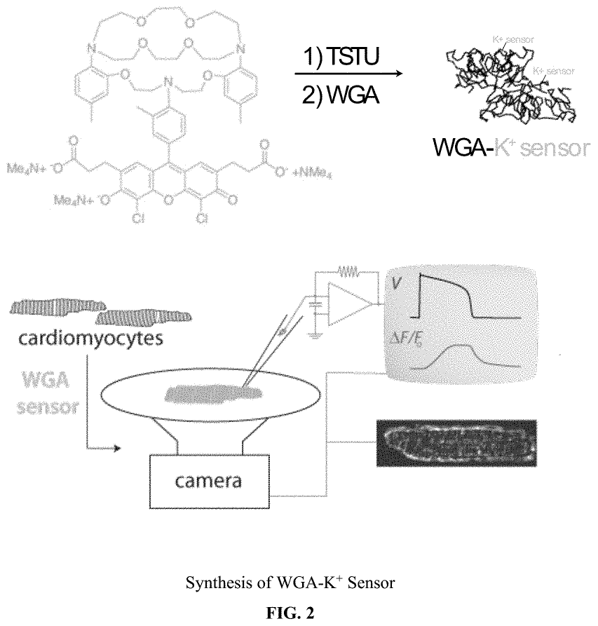

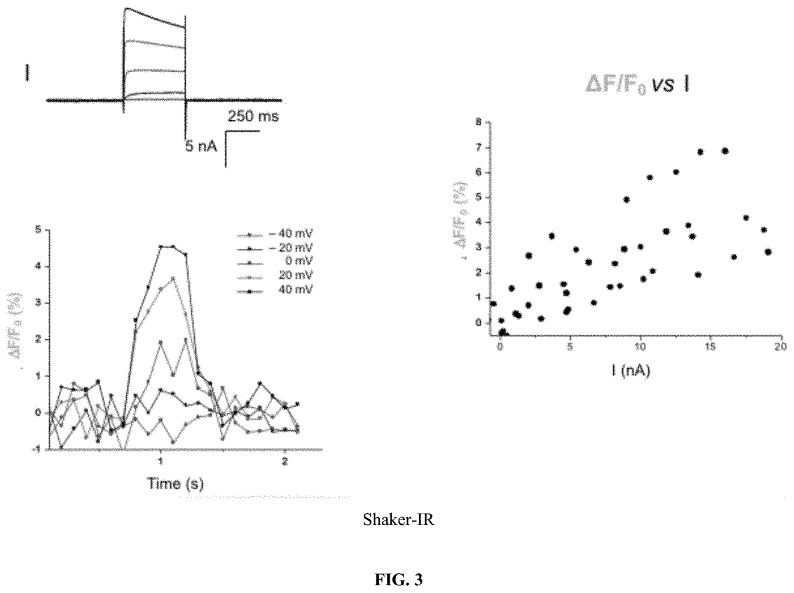

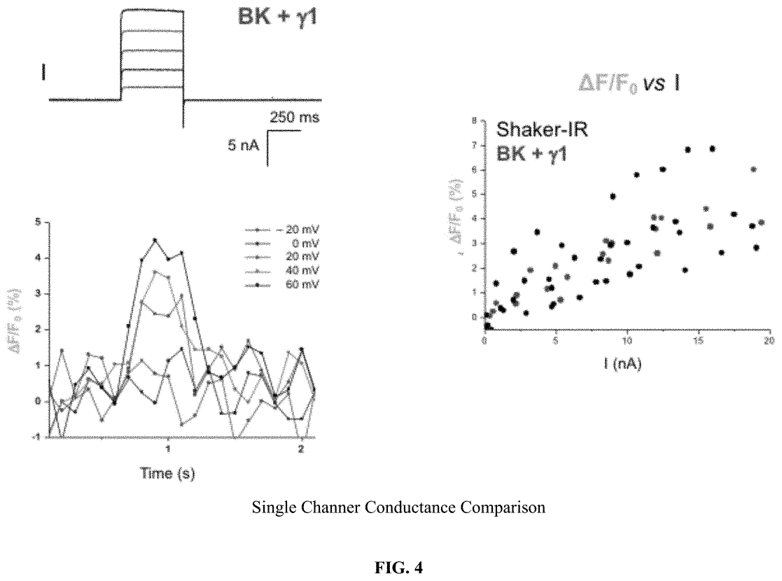

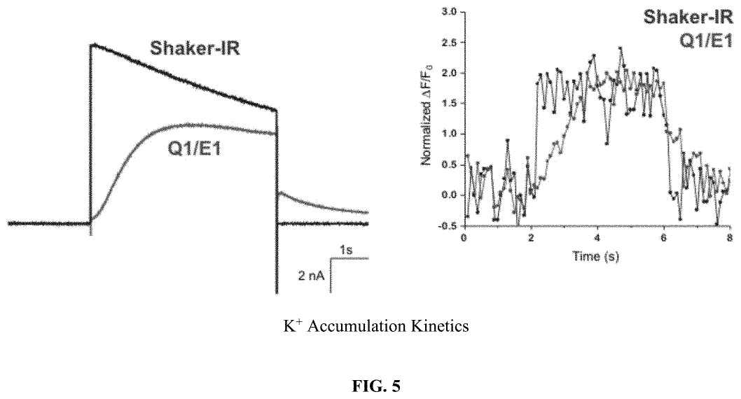

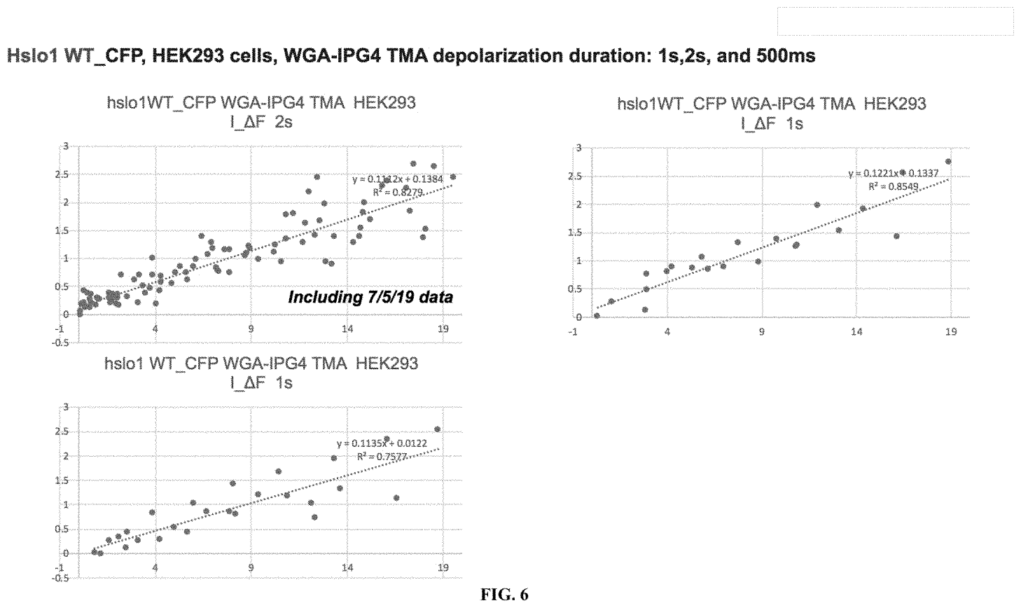

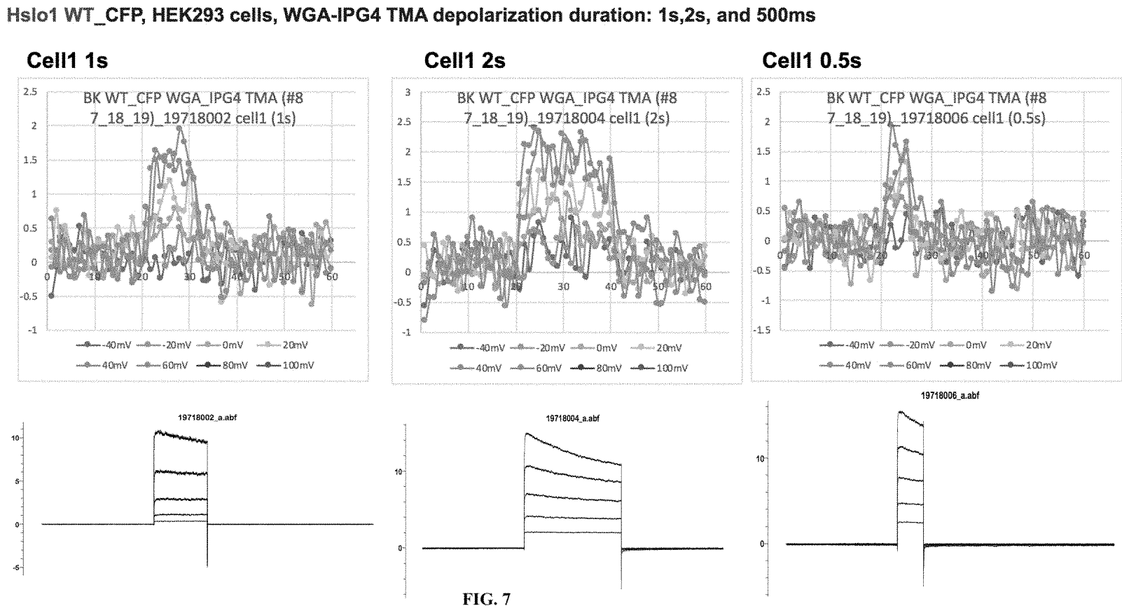

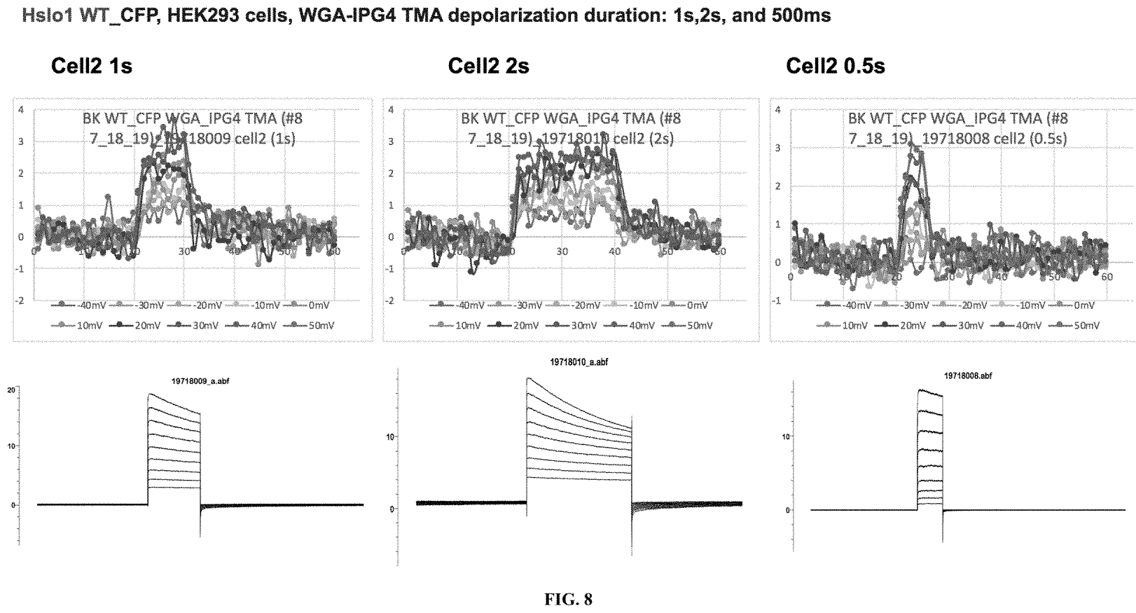

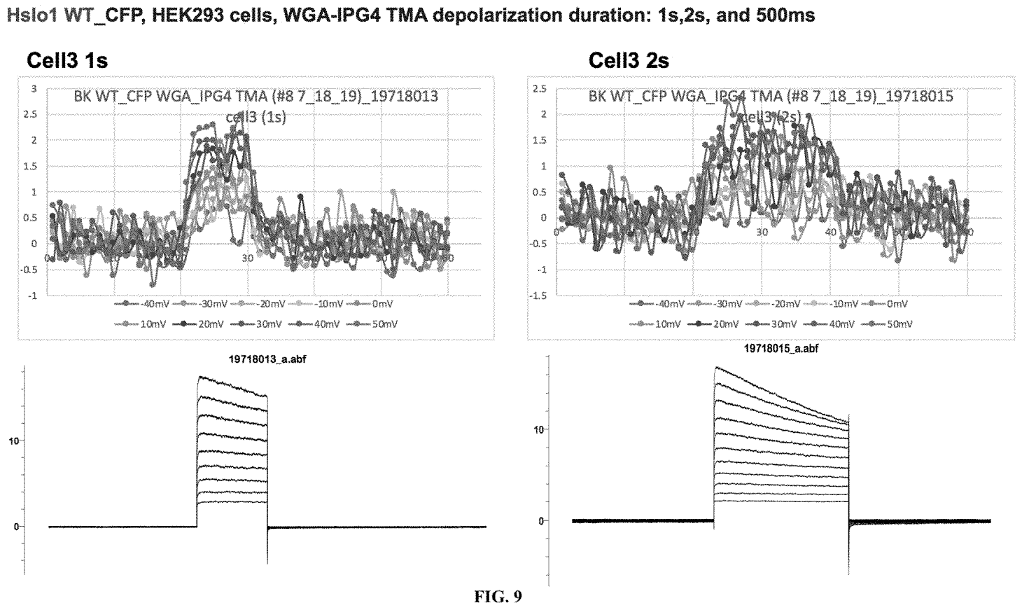

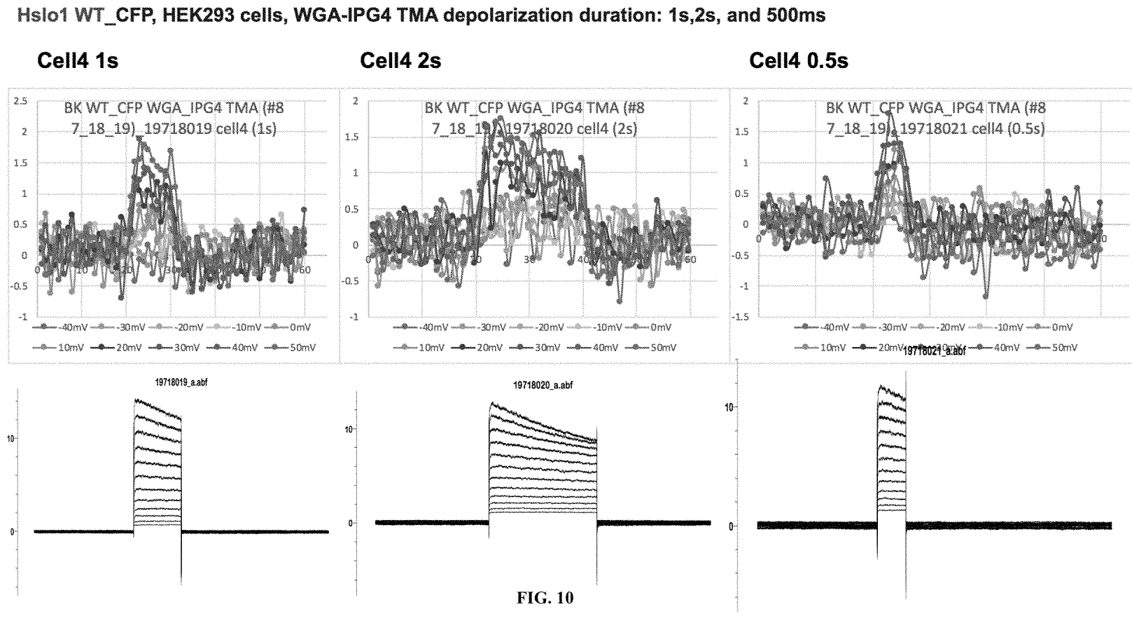

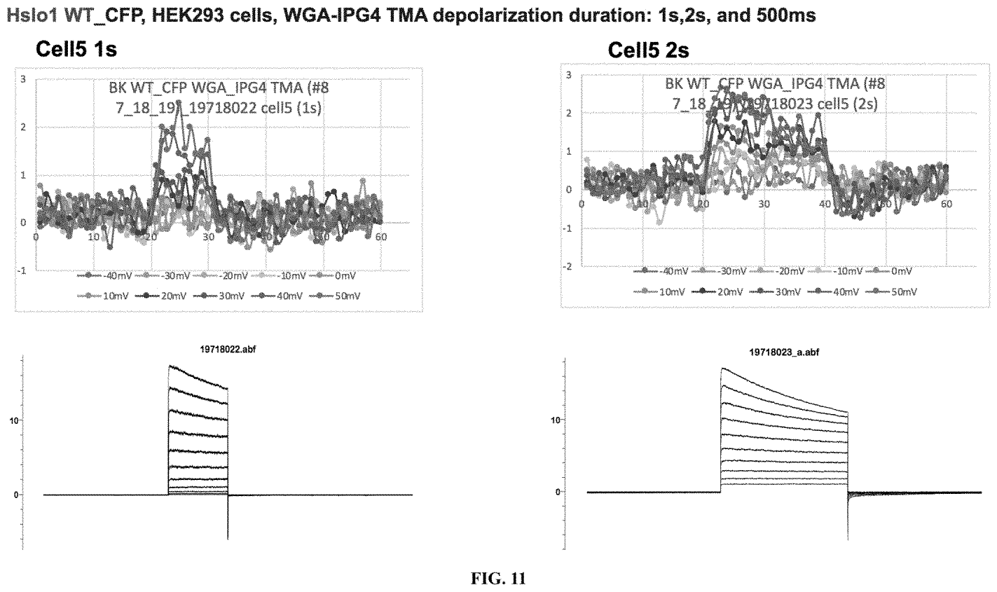

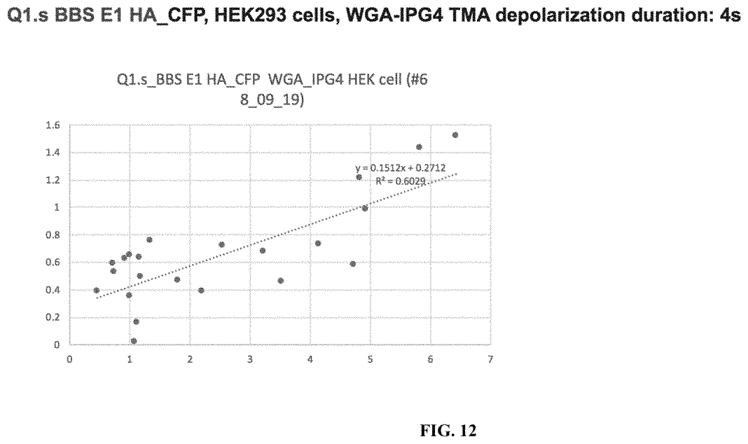

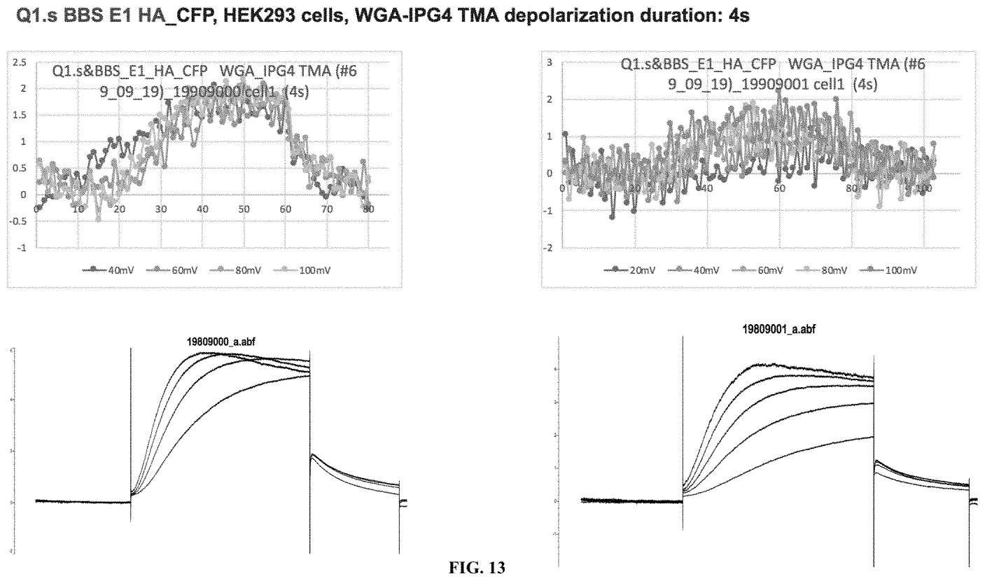

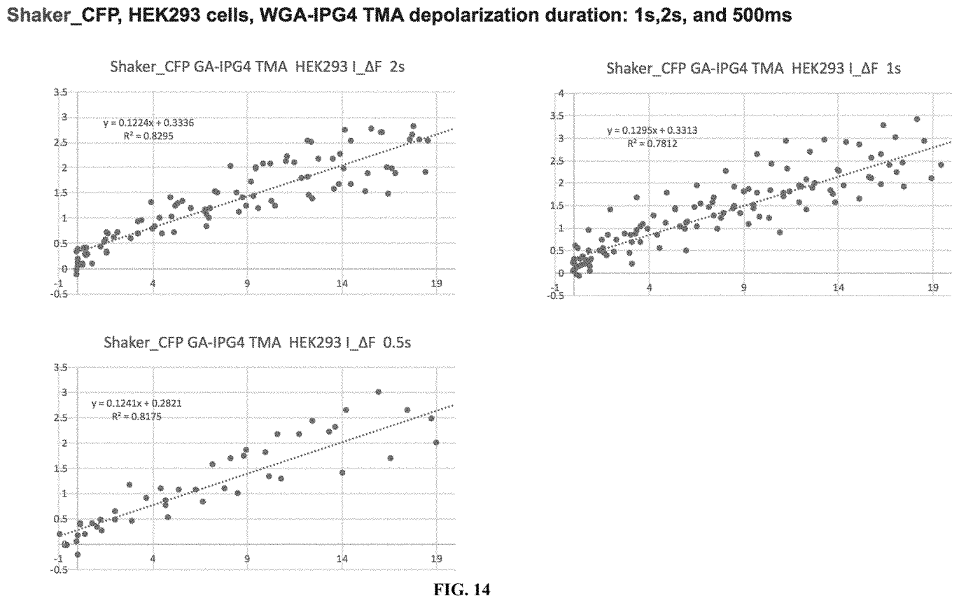

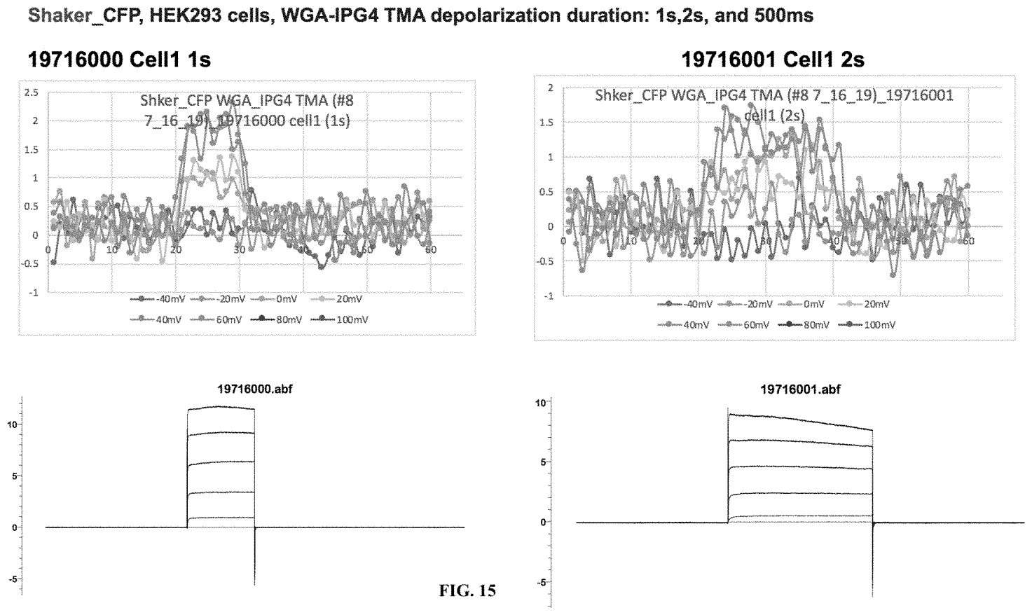

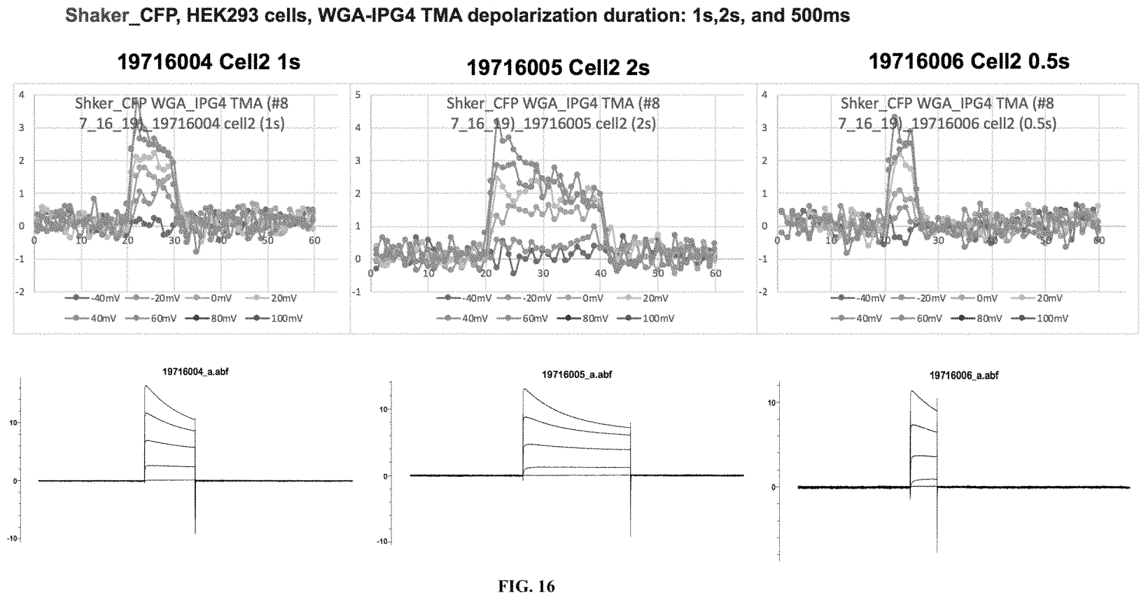

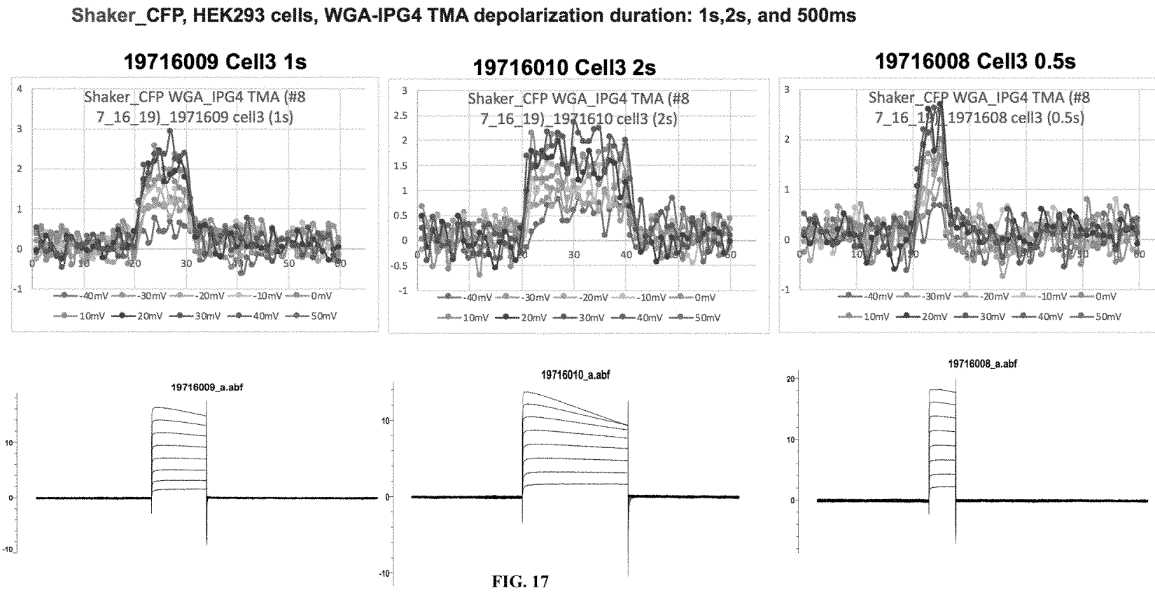

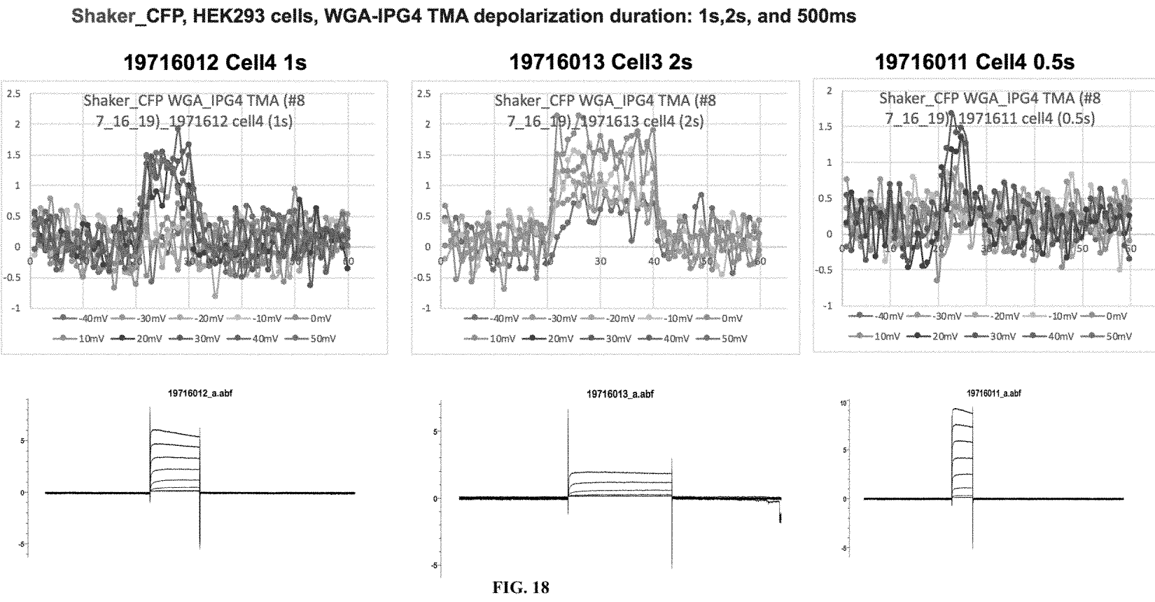

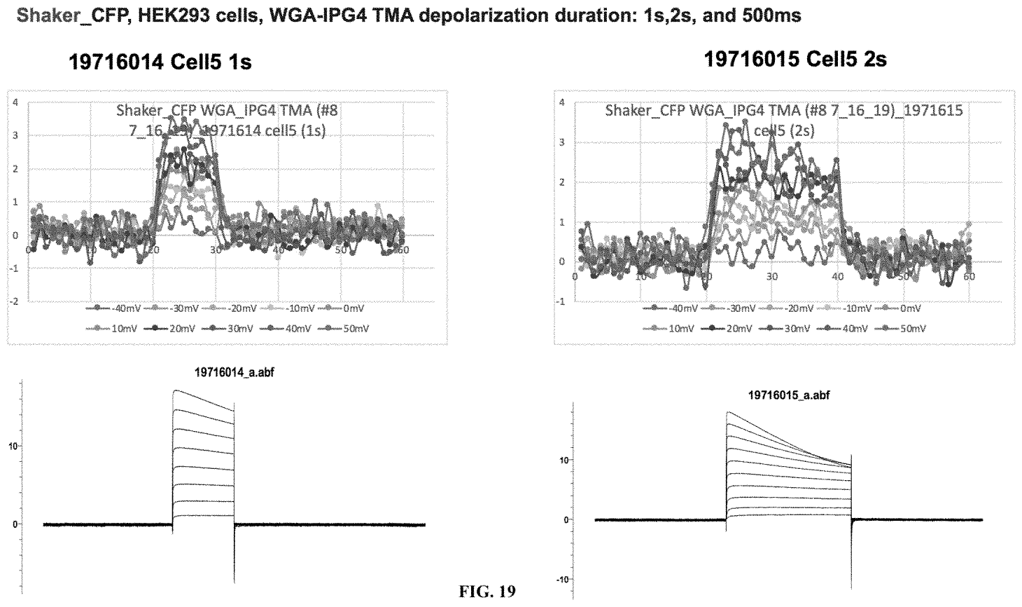

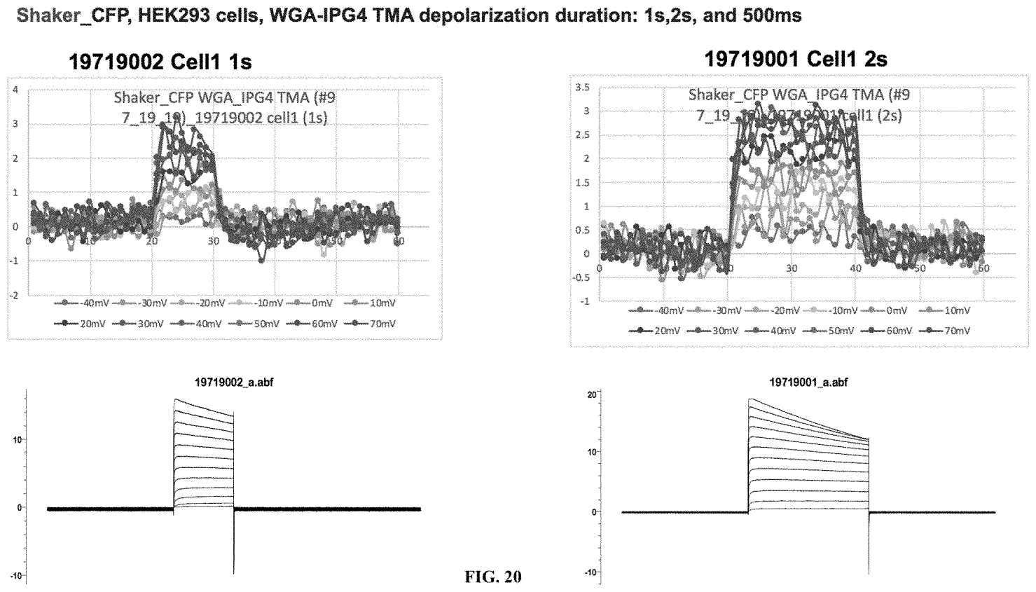

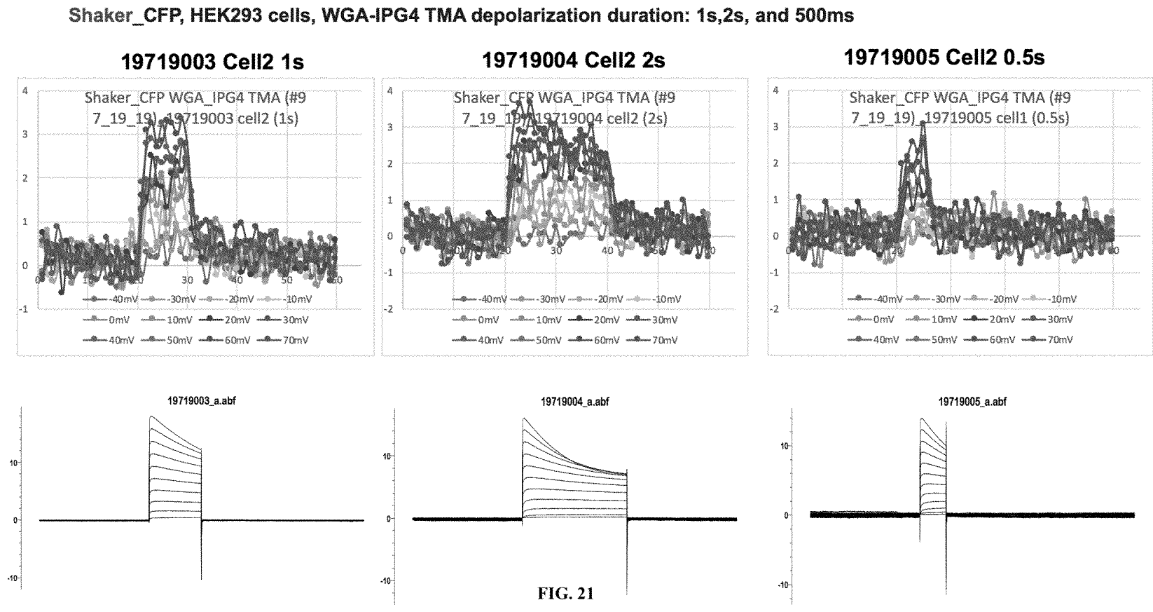

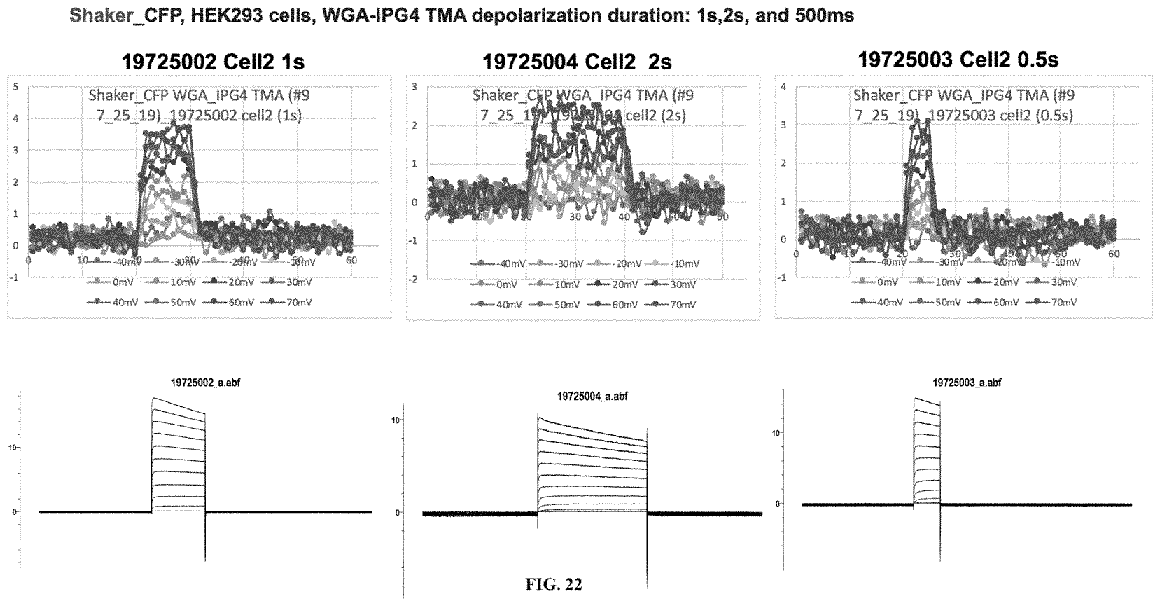

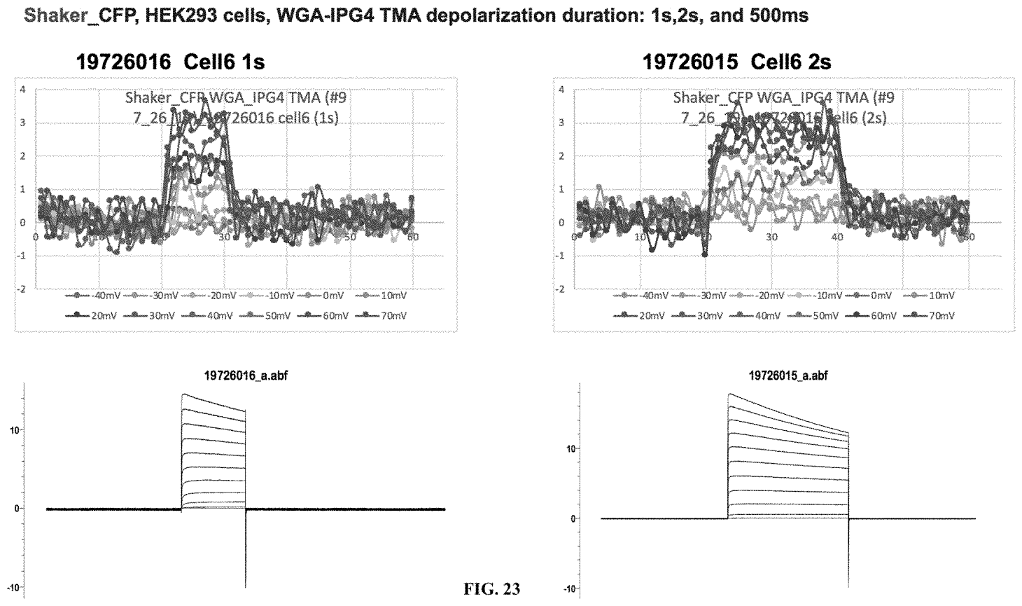

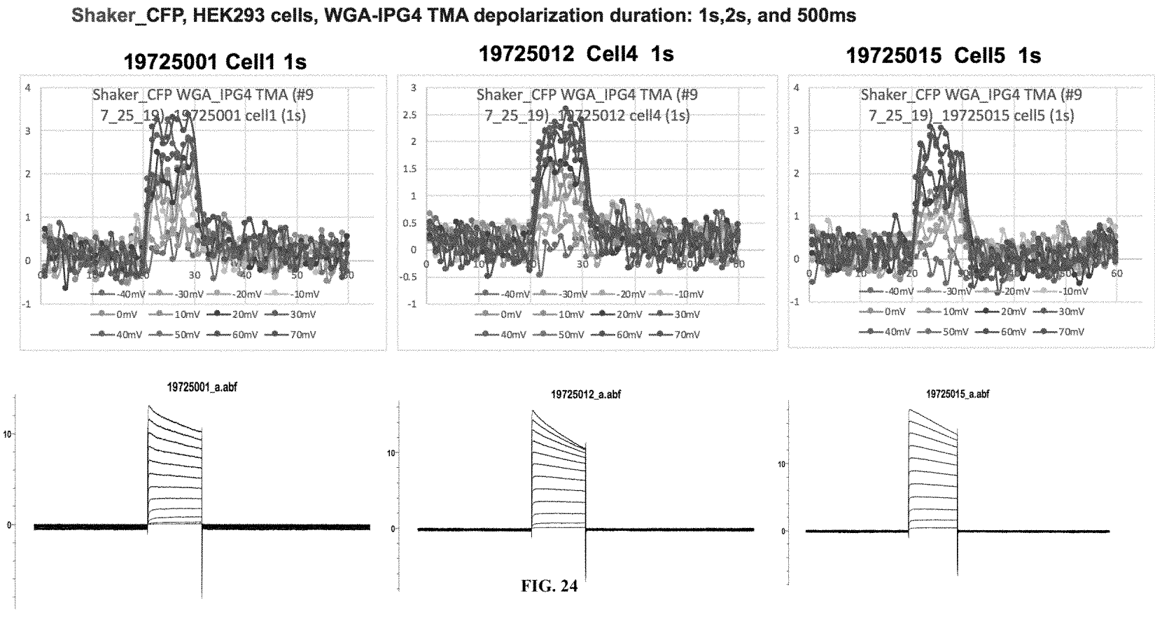

Exemplary compound structures and experimental results are shown in FIGS. 1-5 and in Appendixes A, B and C.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound comprising Wheat Germ Agglutinin (WGA) covalently conjugated to a potassium ion (K⁺) sensor:
WGA-(K⁺sensor)ₙ,
wherein n is an integer from 1 or 2.

2. The compound of claim 1, wherein the K⁺ sensor comprises a fluorophore adapted to exhibiting a change in fluorescent profile and/or intensity upon binding of the sensor to a potassium ion.

3. The compound of claim 1, wherein the K⁺ sensor comprises an ionophore capable of complexing a potassium ion.

4. The compound of claim 3, wherein the ionophore is a crown ether or cryptand, or a derivative thereof.

5. The compound of claim 1, wherein the fluorescent profile comprises an emission in the range from about 500 nm to about 600 nm.

6. The compound of claim 1, wherein the WGA is covalently conjugated to the ionophore.

7. The compound of claim 1, wherein the WGA is covalently conjugated to the potassium ion sensor via an ester bond.

8. The compound of claim 1, wherein the WGA is covalently conjugated to the potassium ion sensor via an amide bond.

9. The compound of claim 1, wherein the fluorophore of the K⁺ sensor comprises the following conjugated system:

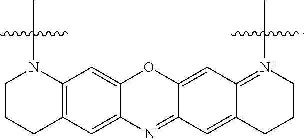 [SMILES: *N1CCCc2cc3c(cc21)Oc1cc2c(cc1=N3)CCC[N+]=2*]

10. The compound of claim 1, wherein the fluorophore of the K⁺ sensor comprises: 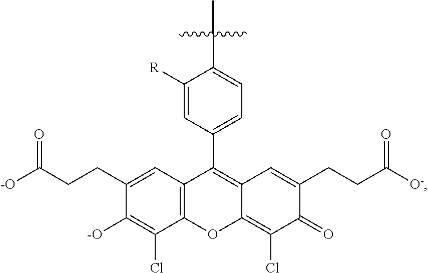
wherein R is a C₁-C₆ alkyl or a C₁-C₆ alkoxy group.

11. The compound of claim 3, wherein the ionophore comprises:

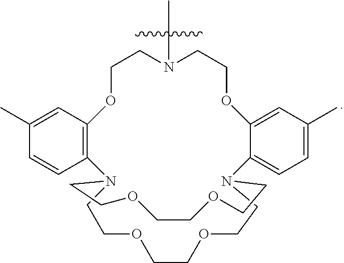

12. The compound of claim 3, wherein the ionophore comprises:

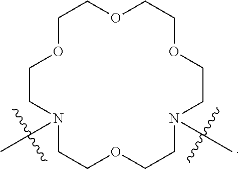 [SMILES: *N1CCOCCOCCOCCN(*)CCOCC1]

13. The compound of claim 1, comprising 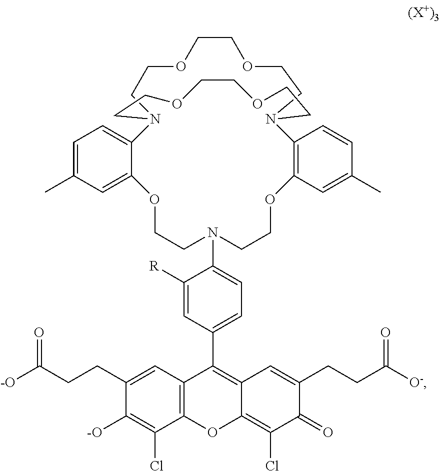
wherein X is a counterion, or a derivative thereof.

14. The compound of claim 1, wherein n is 2.

15. A composition comprising a compound of claim 1.

16. A kit for the assessing extracellular potassium ion, the kit comprising a compound of claim 1.

17. A method for making a compound of claim 1, comprising reacting WGA with a precursor of the potassium ion (K⁺) sensor to form a covalent linkage therebetween.

18. A complex comprising a compound of claim 1 complexed to a K⁺.

19. A composition comprising a complex of claim 18.

20. A method for imaging potassium ion (K⁺), comprising
contacting a sample of cells or tissue with a compound comprising Wheat Germ Agglutinin (WGA) covalently conjugated to a fluorescent potassium ion (K⁺) sensor; and
acquiring one or more fluorescent images of the sample.

21-39. (canceled)
